(19) United States
(12) Patent Application Publication
(10) Pub. No.: US 2002/0133042 A1
(43) Pub. Date: Sep. 19, 2002

(54) Preparation of highly functional aromatic polyisocyanates

(76) Inventors: Imbridt Murrar, Senftenberg (DE); Heinz Plaumann, Flat Rock, MI (US); Jurgen Winkler, Schwarzheide (DE); Marita Schuster, Senftenberg (DE)

Correspondence Address: BASF CORPORATION, LEGAL DEPARTMENT, 1609 BIDDLE AVENUE, WYANDOTTE, MI, 48192, US

(21) Appl. No.: 10/023,353

(22) Filed: Dec. 17, 2001

(30) Foreign Application Priority Data
Dec. 22, 2000 (DE) 10064646.8

(51) Int. Cl. C07C 265/12

(52) U.S. Cl. 560/336

(57) ABSTRACT

Highly functional aromatic polyisocyanates are prepared by reacting aromatic polyisocyanates, if required as a mixture with further mono- and/or polyisocyanates, with addition of catalytic acidic substances and water, by a process wherein the aromatic polyisocyanates used comprise at least one tolylene diisocyanate and the catalytic acidic substance used is at least one alkyl and/or aralkyl phosphate, and the process is carried out in such a way that the water is added with a temperature/time gradient increase from 5 to 60°/hour and until an isocyanate modification of the starting NCO terminal groups from 1 to 80% is established.

The highly functional aromatic polyisocyanates prepared by this process are used for the preparation of polyurethane foams, in particular flexible polyurethane foams.

The present invention relates to a process for the preparation of highly functional aromatic polyisocyanates by reacting aromatic polyisocyanates, if required as a mixture of further mono- and/or polyisocyanates, with addition of catalytic acidic substances and water.

The preparation of highly functional aromatic polyisocyanates by direct reaction of aromatic polyisocyanate in stoichiometric excess with water is sufficiently well known and is widely described in the technical literature (e.g. Kunststoff Handbuch, Vol. 7: Polyurethane, Becker/Braun, 3rd Edition, 1993, Carl Hanser Verlag, Munich, page 76 et seq.) and in patents.

DE-A-2032174 describes the preparation of aromatic polyisocyanate by reacting monomeric polyisocyanate in excess with water in the presence of emulsifiers, such as castor oil polyethylene glycol ether having an OH number of 50 mg KOH/g. The reaction of the isocyanate/urea suspension is effected thermally by stirring for 3 hours at 170° C.

DD-A-151159 claims a process for the preparation of polyisocyanates, in which di- and/or triisocyanates or disubstituted urea isocyanates synthesized from isocyanates are reacted with water in the presence of organotin compounds of the type (R₃Sn)₂O, such as bis(tributyltin) oxide, and, if required, of a solvent at from 50 to 120° C.

DE-A-3526233 describes a process for the preparation of modified polyisocyanates by reacting polyisocyanates in excess with a mixture of amino-containing polyether alcohols or polyester alcohols and water.

GB-A-1078390 discloses the formation of special polyisocyanates directly from diamines and aromatic polyisocyanates by carrying out this reaction in solvents whose boiling point is below the boiling point of the polyisocyanate and removing the solvent by distillation after the reaction.

DE-A-19707576 describes a one-stage process for the preparation of polyisocyanates, in which aromatic diisocyanates are continuously combined with aromatic diamines in a molar ratio of at least 8:1 in a heatable mixing chamber and reacted at above 180° C. and with a residence time of not more than 60 seconds.

PL 134633 describes a process for the preparation of polyisocyanates by reacting polyisocyanates with water in the presence of halogen-substituted esters of phosphoric acid, such as di(β-chloroethyl) phosphate, tri(2,3-dichloropropyl) phosphate or chloro- or bromotrixylyl phosphate, or halogen-containing polymeric esters of phosphoric acid and their use in the preparation of polyurethane foams.

The highly functional polyisocyanates which can be prepared according to the prior art have the following disadvantages: secondary reactions which result in the formation of high molecular weight insoluble polyisocyanate derivatives (also see Saunders, Frisch: Polyurethanes, Chemistry and Technology, Vol. 1 and 2, Interscience, New York 1962, High Polymers, vol. 16), long reaction times and high viscosities occur. In addition, expensive technological process steps, such as the synthesis of special amino-containing polyether alcohols or polyester alcohols as starting materials, special mixed reactor designs and the use of solvents and additional process steps for their removal, are required. Incomplete removal of the solvent results in a deterioration of physical properties of the polyurethane end product, poor mixing, dark products due to high thermal stress, incompatibilities with the A component, phase formation during foaming and hence deterioration of resilience, hardness and load-bearing capacity in the case of flexible polyurethane foams.

It is an object of the present invention to provide a solvent-free process for the preparation of highly functional aromatic polyisocyanates by reacting polyisocyanates and water with addition of catalytic acidic substances, where the modified highly functional polyisocyanates should have medium processing viscosities, improved color and good system compatibility in the A component. During the processing of such components, it should be possible to produce, without phase formation, flexible polyurethane foams of high resilience and high load-bearing capacity, in particular for the use in the automotive industry and furniture industry.

We have found that this object is achieved if the aromatic polyisocyanate used is at least one tolylene diisocyanate and the catalytic acidic substance used is at least one alkyl phosphate and/or aralkyl phosphate and the process is carried out in such a way that the addition of the water is effected with a temperature/time gradient increase from 5 to 60°/hour until an isocyanate modification of the starting NCO terminal groups of from 1 to 80% is established.

The present invention accordingly relates to a process for the preparation of highly functional aromatic polyisocyanates by reacting aromatic polyisocyanates, if required as a mixture with further mono- and/or polyisocyanates, with addition of catalytic acidic substances and water, wherein the aromatic polyisocyanates used comprise at least one tolylene diisocyanate and the catalytic acidic substance used is at least one alkyl phosphate and/or aralkyl phosphate, and the process is carried out in such a way that the addition of water is effected with a temperature/time gradient increase from 5 to 60°/hour until an isocyanate modification of the starting NCO terminal groups of from 1 to 80% is established.

The present invention furthermore relates to the highly functional aromatic polyisocyanates prepared by this process and to their use for the production of polyurethane foams, in particular flexible polyurethane foams.

According to the invention, the aromatic polyisocyanate used is at least one tolylene diisocyanate (also referred to below as TDI). Industrially available TDI is preferably used, for example 2,4′- and 2,6-TDI′ in industrially available isomer ratios, such as TDI 100, TDI 80 or TDI 65. TDI 80 is particularly preferred.

The TDI employed according to the invention can be used as a mixture with further aromatic mono- and/or polyisocyanates and/or aliphatic mono- and/or polyisocyanates and/or cycloaliphatic mono- and/or polyisocyanates.

Examples of further suitable aromatic mono- and/or polyisocyanates are phenyl isocyanate, diphenylmethane 4,4′-, 2,4′- and 2,2′-diisocyanate and the corresponding isomer mixtures, polyphenylpolymethylene polyisocyanates, mixtures of diphenylmethane 4,4′-, 2,4′- and 2,2′-diisocyanates and polyphenylpolymethylene polyisocyanates (crude MDI).

Examples of suitable aliphatic mono- and/or polyisocyanates are alkylene diisocyanates having 4 to 12 carbon atoms in the alkylene radical, such as dodecane 1,12-diisocyanate, 40 2-ethyltetramethylene 1, 4-diisocyanate, 2-methylpentamethylene 1,5-diisocyanate, tetramethylene 1 ,4-diisocyanate and preferably hexamethylene 1,6-diisocyanate.

Cycloaliphatic mono- and/or polyisocyanates which may be used are, for example, cyclohexane 1,3- and -1,4-diisocyanate, 1-isocyanato-3,3,5-trimethyl-5-isocyanatomethylcyclohexane (IPDI), hexahydrotolylene 2,4- and 2,6-diisocyanate and dicyclohexylmethane 4,4′-, 2,2′- and 2,4′-diisocyanate.

Modified aromatic, aliphatic and cycloaliphatic polyisocyanates and prepolymers which are obtained by chemical reaction or organic di- and/or polyisocyanates are also suitable. Examples are di- and/or polyisocyanates containing ester, urea, allophanate, carbodiimide, isocyanurate, uretdione and/or urethane groups. Specific examples are modified diphenylmethane 4,4′-diisocyanate, modified diphenylmethane 4,4′- and 2,4′-diisocyanate mixtures, modified crude MDI or tolylene 2,4- or 2,6-diisocyanate, organic, preferably aromatic, polyisocyanates containing urethane groups and having NCO contents of from 50 to 15, preferably from 31 to 21, % by weight, based on the total weight, for example reaction products of low molecular diols, triols, dialkylene glycols, trialkylene glycols or polyoxyalkylene glycols having molecular weights of up to 6000, in particular up to 1500, it being possible for these di- and polyoxyalkylene glycols to be used individually or as a mixture. Examples are diethylene and dipropylene glycol, polyoxyethylene, polyoxypropylene and polyoxypropylenepolyoxyethene glycols and corresponding triols and/or tetrols. Also suitable are NCO-containing prepolymers having NCO contents of from 25 to 3.5, preferably from 21 to 14, % by weight, based on the total weight, prepared from polyesterpolyols and/or polyetherpolyols and diphenylmethane 4,4′-diisocyanate, mixtures of diphenylmethane 2,4′- and 4,4′-diisocyanate, tolylene 2,4′- and/or 2,6′-diisocyanates or crude MDI.

The aromatic, aliphatic, cycloaliphatic and modified mono- and/or polyisocyanate can be used individually or in any desired mixtures with one another. If such isocyanates are used in addition to TDI, they are preferably used in amounts of not more than 85, advantageously from 5 to 60, % by weight.

The catalytic acidic substances used are, for example, the following: organotin compounds, such as dibutyltin dilaurate, tin(II) octoate, butyltin trichloride, dibutyltin dichloride, triethyltin chloride, dibutyltin diacetate, dimethyltin diethylhexanoate and mono-, di- and/or trialkyl(aryl) esters of phosphoric acid, such as bis(2-ethylhexyl) phosphate, dihexyl decyl phosphate, dibutyl phosphate, dipropyl phosphate and particularly preferably bis(2-ethylhexyl) phosphate. The catalytic acidic substances can be used individually in any desired mixtures with one another.

The total amount of catalytic acidic substances used is preferably from 0.001 to 3.0, in particular from 0.01 to 1.0, % by weight, based in each case on the weight of the total formulation.

The reactant water is used in the form of free water, substances releasing water of crystallization, water-eliminating substances or steam. The various forms of water used can also be combined with one another. The free water or steam is preferably used.

Examples of suitable substances releasing water of crystallization are aquocomplexes, such as copper sulfate pentahydrate, Cd SO₄V8/3 H₂O, crystalline sodium carbonate, iron sulfate heptahydrate and potassium aluminum sulfate dodecahydrate.

The water-eliminating substances used may be, for example, tert-butanol or salicylic acid.

The total amount of water used is preferably from 0.005 to 0.03, particularly preferably from 0.01 to 0.026, g/g of polyisocyanate.

According to the invention, the process is carried out in such a way that the water is added with a temperature/time gradient increase from 5 to 60°/hour until an isocyanate modification of the starting NCO terminal groups of from 1 to 80% is established. The process can be varied with regard to the sequence of the initial introduction or addition of the starting components.

According to an advantageous process variant, the tolylene diisocyanate or the isocyanate mixture and the catalytic acidic substances are initially taken and the water is then metered in with the novel temperature/time gradient increase from 5 to 60°/h, preferably from 9 to 40°/h, until an isocyanate modification of the starting NCO terminal groups of from 1 to 80%, preferably from 8 to 60%, has been established.

It is also advantageous if the tolylene diisocyanate or the isocyanate mixture or the catalytic acidic substance is first initially taken, the other respective components are then metered in within from 0.01 to 2.5, preferably from 0.03 to 1.5, hours with a temperature increase of up to 100°, preferably from 35 to 85°, and the addition of water is then carried out according to the temperature/time regime prescribed according to the invention, preferably with a temperature/time gradient increase from 9 to 40°/h, until an isocyanate modification of the starting NCO terminal groups of preferably from 8 to 60% has been established.

The highly functional aromatic polyisocyanates prepared according to the invention preferably contain from 15 to 95, in particular from 20 to 85, parts by weight of the modified highly functional polyisocyanate and from 85 to 5, in particular from 80 to 15, parts by weight of tolylene diisocyanate and/or monomers and/or polymers of the isocyanate mixture used. The proportion of highly functional aromatic polyisocyanates depends on the isocyanate modification established.

The highly functional aromatic polyisocyanates prepared according to the invention preferably contain ≦8 parts by weight of urea and higher molecular weight oligomers and ≦2 parts by weight of uretdione and higher molecular weight oligomers.

The novel highly functional aromatic polyisocyanates preferably contain from 35 to 39% by weight of free NCO groups and have a viscosity of from 3 to 6000, in particular from 50 to 150, mpa•s, measured in each case at 25° C., a mean functionality of from 2.6 to 3.2, an average molecular weight of from 300 to 600, in particular from 425 to 475, g/mol and an iodine color number of more than 10, advantageously not more than 2.

The novel process has the following advantages: highly functional aromatic polyisocyanates having improved color and a medium viscosity can surprisingly be prepared with a high space/time yield by a solvent-free preparation process. A particular advantage of the process is the good long-term stability of the resulting highly functional aromatic polyisocyanates, which guarantee optimum processing. The excellent low-temperature stability of the product is very particularly advantageous. The novel highly functional aromatic polyisocyanates have a melting range of from −20 to +11° C. and a glass transition temperature of from −80 to −70° C.

Owing to the medium viscosity, any desired isocyanate mixtures can be prepared and can be processed on conventional foaming machines.

When the highly functional aromatic polyisocyanates are used, it is surprisingly possible to prepare and to process polyurethane systems without separation and with system compatibility with all raw materials used. Highly functional aromatic polyisocyanates which can be used in polyurethane applications, preferably in foams, particularly advantageously in flexible polyurethane foams for the automotive industry and furniture industry are thus provided.

For the production of flexible polyurethane foams, the novel highly functional aromatic polyisocyanates, if required as a mixture with further organic and/or modified organic polyisocyanates (a), are reacted with compounds (b) having hydrogen atoms reactive toward isocyanates, in the presence of water and/or other blowing agents (c), catalysts (d) and, if required, further assistants and additives (e) by conventional processes.

Regarding the starting components, the following may be stated specifically:

If further organic and/or modified organic polyisocyanates (a) are used in addition to the novel highly functional aromatic polyisocyanates, the aliphatic, cycloaliphatic, araliphatic and preferably aromatic isocyanates known per se and having a functionality of ≧2, as described by way of example further above, are suitable.

If further polyisocyanates are concomitantly used, they are employed in an amount of not more than 95, preferably from 15 to 85, % by weight, based on the total weight of the polyisocyanates used.

The compounds (b) used which have hydrogen atoms reactive toward isocyanates are expediently those having a functionality of from 2 to 8, preferably from 2 to 3, and an average molecular weight of from 300 to 8000, preferably from 300 to 5000.

For example, it is possible to use polyols selected from the group consisting of the polyetherpolyols, polyesterpolyols, polythioetherpolyols, polyesteramides, hydroxyl-containing polyacetals and hydroxyl-containing aliphatic polycarbonates, or mixtures of at least two of said polyols. The hydroxyl number of the polyhydroxyl compounds is as a rule from 20 to 80, preferably from 28 to 60. For example, polyetherpolyamines may also be used.

The polyetherpolyols used are prepared by known processes, for example by anionic polymerization using alkali metal hydroxides, e.g. sodium hydroxide or potassium hydroxide, or alkali metal alcoholates, e.g. sodium methylate, potassium methylate or potassium isopropylate, as catalysts and with the addition of at least one initiator which contains from 2 to 8, preferably 2 to 3, bonded hydrogen atoms per molecule, or by cationic polymerization using Lewis acids, such as antimony pentachloride, boron fluoride etherate, etc., or bleaching earths as catalysts or from one or more alkylene oxides having 2 to 4 carbon atoms in the alkylene radical by double metal cyanide catalysis. For specific intended uses, monofunctional initiators may also be incorporated into the polyether structure.

Suitable alkylene oxides are, for example, tetrahydrofuran, 1,3-propylene oxide, 1,2- and 2,3-butylene oxide, styrene oxide and preferably ethylene oxide and 1,2-propylene oxide. The alkylene oxides may be used individually, directly in succession or as mixtures.

Examples of suitable initiator molecules are water, organic dicarboxylic acids, such as succinic acid, adipic acid, phthalic acid and terephthalic acid, aliphatic and aromatic, unsubstituted or N-monoalkyl-, N,N-dialkyl- and N,N′-dialkyl-substituted diamines having 1 to 4 carbon atoms in the alkyl radical, such as unsubstituted or monoalkyl- and dialkyl-substituted ethylenediamine, diethylenetriamine, triethylenetetramine, 1,3-propylenediamine, 1,3- and 1,4-butylenediamine, 1,2-, 1,3-, 1,4-, 1,5- and 1,6-hexamethylenediamine, phenylenediamine, 2,3′-, 2,4′- and 2,6′-tolylenediamine and 4,4′, 2,4′- and 2,2′-diaminodiphenylmethane. Other suitable initiator molecules are alkanolamines, e.g. ethanolamine and N-methyl- and N-ethylethanolamine, dialkanolamines, e.g. diethanolamine and N-methyl- and N-ethyldiethanolamine, and trialkanolamines, e.g. triethanolamine, and ammonia. Polyhydric, in particular dihydric and/or trihydric, alcohols, such as ethanediol, 1,2- and 2,3-propanediol, diethylene glycol, dipropylene glycol, 1,4-butanediol, 1,6-hexanediol, glycerol, trimethylolpropane and pentaerythritol, are preferably used.

The polyetherpolyols, preferably polyoxypropylenepolyols and polyoxypropylenepolyoxyethylenepolyols, have a functionality of, preferably, from 2 to 8, in particular from 2 to 3, and molecular weights of from 300 to 8000, preferably from 300 to 6000, and in particular from 1000 to 5000, and suitable polyoxytetramethylene glycols have a molecular weight of about 3500.

Other suitable polyetherpolyols are polymer-modified polyetherpolyols, preferably graft polyetherpolyols, in particular those based on styrene and/or on acrylonitrile, which are prepared by in situ polymerization of acrylonitrile, styrene or preferably mixtures of styrene and acrylonitrile, for example in a weight ratio of from 90:10 to 10:90, preferably from 70:30 to 30:70, expediently in the abovementioned polyetherpolyols analogously to the information in German patents 1111394, 1222669 (U.S. Pat. Nos. 3,304,273, 3,383,351, 3,523,093), 1,152,536 (GB 1040452) and 1,152,537 (GB 987618), and polyetherpolyol dispersions which, as the disperse phase, usually contain from 1 to 50, preferably from 2 to 25, % by weight of, for example, polyureas, polyhydrazides, polyurethanes containing bonded tertiary amino groups and/or melamine. Such polyetherpolyols are described, for example, in EP-B-011752 (U.S. Pat. No. 4,304,708), U.S. Pat. No. 4,374,209 and DE-A-3231497.

The polyetherpolyols may be used individually or in the form of mixtures.

In addition to the polyetherpolyols described, it is also possible to use, for example, polyetherpolyamines and/or further polyols selected from the group consisting of the polyesterpolyols, polythioetherpolyols, polyesteramides, hydroxyl-containing polyacetals and hydroxyl-containing aliphatic polycarbonates or mixtures of at least two said polyols. The hydroxyl number of the polyhydroxyl compounds is as a rule from 20 to 80, preferably from 28 to 56.

Suitable polyesterpolyols can be prepared, for example, from organic dicarboxylic acids of 2 to 12 carbon atoms, preferably aliphatic dicarboxylic acids of 4 to 6 carbon atoms, and polyhydric alcohols, preferably diols, of 2 to 12, preferably 2 to 6, carbon atoms, by conventional processes. Usually, the organic polycarboxylic acids and/or derivatives thereof and polyhydric alcohols are subjected to polycondensation, advantageously in a molar ratio of from 1:1 to 1.8, preferably from 1:1.05 to 1.2, in the absence of a catalyst or preferably in the presence of an esterification catalyst, expediently in an atmosphere comprising inert gas, e.g. nitrogen, carbon monoxide, helium, argon, etc., in the melt at from 150 to 250° C., preferably from 180 to 220° C., if required under reduced pressure, to the desired acid number, which is advantageously less than 10, preferably less than 2.

Examples of suitable hydroxyl-containing polyacetals are the 40 compounds which can be prepared from glycols, such as diethylene glycol, triethylene glycol, 4,4′-dihydroxyethoxydiphenyldimethylmethane, hexanediol, and formaldehyde. Suitable polyacetals can also be prepared by polymerizing cyclic acetals. Suitable hydroxyl-containing polycarbonates are those of the type known per se, which can be prepared, for example, by reacting diols, such as 1,3-propanediol, 1,4-butanediol and/or 1,6-hexanediol, diethylene glycol, triethylene glycol or tetraethylene glycol, with diaryl carbonates, e.g. diphenyl carbonate, or phosgene. The polyesteramides include, for example, the predominantly linear condensate obtained from polybasic, saturated and/or unsaturated carboxylic acids or their anhydrides and polyhydric saturated and/or unsaturated amino alcohols or mixtures of polyhydric alcohols and amino alcohols and/or polyamines. Suitable polyetherpolyamines can be prepared from the abovementioned polyetherpolyols by known processes. The cyanoalkylation of polyoxyalkylenepolyols and subsequent hydrogenation of the resulting nitrile (U.S. Pat. No. 3,267,050) or the partial or complete amination of polyoxyalkylenepolyols with amines or ammonia in the presence of hydrogen and catalysts (DE-A-1215373) may be mentioned by way of example.

The flexible polyurethane foams can be produced in the presence or absence of chain extenders and/or crosslinking agents, but, as a rule, these are not required. The chain extenders and/or crosslinking agents used are diols and/or triols having molecular weights of less than 400, preferably from 60 to 300. For example, aliphatic, cycloaliphatic and/or araliphatic diols of 2 to 14, preferably 4 to 10, carbon atoms, e.g. ethylene glycol, 1,3-propanediol, 1,10-decanediol, o-, m- and p-dihydroxycyclohexane, diethylene glycol, dipropylene glycol and preferably 1,4-butanediol, 1,6-hexanediol and bis(2-hydroxyethyl)hydroquinone, triols, such as 1,2,4- and 1,3,5-trihydroxycyclohexane, triethanolamine, diethanolamine, glycerol and trimethylolpropane and low molecular weight hydroxyl-containing polyalkylene oxides based on ethylene oxide and/or 1,2-propylene oxide and the abovementioned diols and/or triols are suitable as initiator molecules.

If chain extenders, crosslinking agents or mixtures thereof are used for the production of the polyurethane foams, they are expediently employed in an amount of up to 10% by weight, based on the weight of the polyol compounds.

The compounds of component (b) can be used individually or in the form of mixtures.

The chlorofluorohydrocarbons (CFHC) and highly fluorinated and/or perfluorinated hydrocarbons generally known from polyurethane chemistry can be used as blowing agents (c). However, the use of these substances is greatly restricted or has been completely discontinued for ecological reasons. In addition to chlorofluorohydrocarbons and fluorohydrocarbons, in particular aliphatic and/or cycloaliphatic hydrocarbons, in particular pentane and cyclopentane, or acetals, e.g. methylal, are possible alternative blowing agents. These physical blowing agents are usually added to the polyol component of the system. However, they may also be added in the isocyanate component or in both the polyol component and the isocyanate component. They can also be used together with highly fluorinated and/or perfluorinated hydrocarbons, in the form of an emulsion of the polyol component. Where used, the emulsifiers are usually oligomeric acrylate which contain polyoxyalkylene and fluoroalkane radicals bonded as side groups and have a fluorine content of from about 5 to 30% by weight. Such products are sufficiently well known from plastics chemistry, for example EP-A-0351614. The amount of blowing agent or of a mixture of blowing agents which is used is from 1 to 25, preferably from 1 to 15, % by weight, based in each case on the total weight of the components (b) to (c).

It is also possible and usual to add water in an amount of from 2 to 8, preferably from 2.5 to 4.0, % by weight, based on the total weight of the components (b) to (e), as blowing agent to the polyol component. The addition of water can be effected in combination with the use of the other blowing agents described.

The catalysts (d) used for the preparation of the flexible polyurethane foams are in particular compounds which greatly accelerate the reaction of the reactive hydrogen atoms, in particular of hydroxyl-containing compounds of components (b), (c) and (d), with the organic, unmodified or modified polyisocyanates (a). Organometallic compounds, preferably organotin compounds, such as tin(II) salts of organic carboxylic acids, e.g. tin(II) acetate, tin(II) octanoate, tin(II) ethylhexanoate and tin(II) laurate, and the dialkyltin(IV) salts of organic carboxylic acids, e.g. dibutyltin diacetate, dibutyltin dilaurate, dibutyltin maleate and dioctyltin diacetate, are suitable. The organometallic compounds are used alone or preferably in combination with strongly basic amines. Examples are amidines, such as 2,3-dimethyl-3,4,5,6-tetrahydropyrimidine, tertiary amines, such as triethylamine, tributylamine, dimethylbenzylamine, N-methyl-, N-ethyl- and N-cyclohexylmorpholine, N,N,N′,N′-tetramethylethylenediamine, N,N,N′,N′-tetramethylbutanediamine, N,N,N′,N′-tetramethyl-1,6-hexanediamine, pentamethyldiethylenetriamine, tetramethyldiaminoethyl ether, bis(dimethylaminopropyl)urea, dimethylpiperazine, 1,2-dimethylimidazole, 1-azabicyclo[3.3.0]octane and preferably 1,4-diazabicyclo[2.2.2]octane, and aminoalkanol compounds, such as triethanolamine, triisopropanolamine, N-methyl- and N-ethyldiethanolamine and dimethylethanolamine.

Other suitable catalysts are tris(dialkylaminoalkyl)-s-hexahydrotriazines, in particular tris(N,N-dimethylaminopropyl)-s-hexahydrotriazine, tetraalkylammonium hydroxides, such as tetramethylammonium hydroxide, alkali metal hydroxides, such as sodium hydroxide, and alkali metal alcoholates, such as sodium methylate and potassium isopropylate, and alkali metal salts of long-chain fatty acids of 10 to 20 carbon atoms which may have OH side groups. From 0.001 to 5, in particular from 0.05 to 2, % by weight, based on the weight of the components (b) to (f), of catalyst or catalyst combination are preferably used.

If required, further assistants and/or additives (e) may be incorporated into the reaction mixture for the production of the novel flexible polyurethane foams. Examples of flameproofing agents, stabilizers, fillers, dyes, pigments and hydrolysis stabilizers and fungistatic and bacteriostatic substances.

Suitable flameproofing agents are, for example, tricresyl phosphate, tris(2-chlorethyl) phosphate, tris(2-chloropropyl) phosphate, tetrakis(2-chloroethyl) ethylene diphosphate, dimethyl methanephosphonate, diethyl diethanolaminomethylphosphonate and commercial halogen-containing polyol flameproofing agents. In addition to the abovementioned halogen-substituted phosphates, inorganic or organic flameproofing agents, such as red phosphorus, hydrated alumina, antimony trioxide, arsenic oxide, ammonium polyphosphate and calcium sulfate, expanded graphite or cyanuric acid derivatives, e.g. melamine, or mixtures of at least two flameproofing agents, e.g. ammonium polyphosphates and melamine and, if required, corn starch or ammonium polyphosphate, melamine and expanded graphite and/or, if required, aromatic polyesters, can also be used for flameproofing the polyisocyanate polyadducts. The addition of melamine proves to be particularly effective. In general, it has proven expedient to use from 5 to 50, preferably from 5 to 25, parts by weight of said flameproofing agent per 100 parts by weight of the components (b) to (e).

The stabilizers used are in particular surfactants, i.e. compounds which assist the homogenization of the starting materials and may also be suitable for regulating the cell structure of the plastics. Examples are emulsifiers, such as sodium salts or of castor oil sulfates or fatty acids and salts of fatty acids with amines, for example the salt of oleic acid with diethylamine, of stearic acid with diethanolamine and of ricinoleic acid with diethanolamine, salts of sulfonic acids, for example alkali metal or ammonium salts of dodecylbenzenedisulfonic acid or dinaphthylmethanedisulfonic acid and ricinoleic acid; foam stabilizers such as siloxane oxyalkylene copolymers and other organopolysiloxanes, oxyethylated alkylphenols, oxyethylated fatty alcohols, liquid paraffins, castor oil esters or ricinoleic esters, Turkey red oil and groundnut oil, and cell regulators, such as paraffins, fatty alcohols and dimethylpolysiloxanes. Predominantly used stabilizers are organopolysiloxanes which are water-soluble. These are polydimethylsiloxane radicals onto which a polyether chain comprising ethylene oxide and propylene oxide has been grafted. The surfactants are usually used in amounts of from 0.01 to 5 parts by weight, based on 100 parts by weight of the components (b) to (e).

Fillers, in particular reinforcing fillers, are to be understood as meaning the conventional organic and inorganic fillers, reinforcing agents, weighting compositions, compositions for improving the abrasion behavior in surface coatings, coating materials, etc. Specific examples are inorganic fillers, such as silicate minerals, for example sheet silicates, such as antigorite, serpentin, hornblendes, amphiboles, chrysotile and talc, metal oxides, such as kaolin, aluminas, titanium oxides and iron oxides, metal salts, such as chalk, barite and inorganic pigments, such as cadmium sulfide and zinc sulfide, and glass, etc. Kaolin (china clay), aluminum silicate and coprecipitates of barium sulfate and aluminum silicate, and natural and synthetic fibrous minerals, such as wollastonite, metal fibers and in particular glass fibers of various lengths, which may be sized, are preferably used. Examples of suitable organic fillers are carbon, rosin, cyclopentadienyl resins and graft polymers as well as cellulose fibers, polyamide, polyacrylonitrile, polyurethane and polyester fibers based on aromatic and/or aliphatic dicarboxylic esters and in particular carbon fibers. The inorganic and organic fillers may be used individually or as mixtures and are advantageously incorporated into the reaction mixture in amounts of from 0.5 to 50, preferably from 1 to 40, % by weight, based on the weight of the components (a) to (e), but the content of mats, nonwovens and woven fabrics of natural and synthetic fibers may reach values of up to 80.

Further information on the abovementioned other conventional assistants and additives are to be found in the technical literature, for example the monograph by Saunders, Frisch: Polyurethanes, Chemistry and Technology, Vol. 1 and 2, Interscience Publishers, New York 1962, High Polymers, Vol. 16, or the above-cited Kunststoffhandbuch, Vol. 7: Polyurethane, Hanser-Verlag Munich, Vienna, 1st to 3rd Edition.

For the production of the novel foams, the individual components are reacted in amounts such that the ratio of the number of equivalents of the NCO groups of the polyisocyanates to the sum of the reactive hydrogen atoms for the components (b) to (e) is from 0.75:1 to 1.25:1, preferably from 0.90:1 to 1.15:1.

Polyurethane foams obtained using novel process are advantageously produced by the one-shot method, for example with the aid of the high pressure or low pressure technique in open or closed molds, for example metallic molds. The continuous application of the reaction mixture to suitable belt lines is also usual for the production of slabstock foams.

It has proven particularly advantageous to employ the two-component process and to combine the components (b) to (e) into a polyol component, often also referred to as component A, and to use the polyisocyanate and, if required, blowing agent (c) as the isocyanate component, often also referred to as component B.

The starting components are mixed at from 15 to 90° C., preferably from 20 to 60° C., and in particular from 20 to 35° C., and introduced into the open mold or, if necessary under suitable atmospheric pressure, into the closed mold or, in a continuous workstation, are applied to a belt which takes up the reaction material. The mixing can be carried out mechanically by means of a stirrer, by means of a stirring screw or by high-pressure mixing in a nozzle. The mold temperature is expediently from 20 to 110° C., preferably from 30 to 60° C., and in particular from 35 to 55° C.

Using the highly functional aromatic polyisocyanates prepared according to the invention, flexible polyurethane foams having a density of from 30 to 60 kg/m³ in combination with a total water content of from 2.8 to 4.0 parts by weight and an isocyanate index of from 75 to 115 are obtained.

The flexible polyurethane foams produced have a high load-bearing capacity and are optically suitable for high-stress applications in the automotive industry and furniture industry. It has surprisingly been found that the tear propagation strength of the flexible polyurethane foams can be improved by using the highly functional aromatic polyisocyanates.

The examples which follow illustrate the invention.

The reactions described in the use example were carried out in a 50 l reactor having a stirrer, water metering connection and connection for blanketing with nitrogen (3 bar).

### Examples 1-3

TDI 80 (BASF: Lupranat® T 80 A) was initially taken at 20° C. and the catalytic acidic substance bis(2-ethylhexyl) phosphate was metered in over a period of up to 0.5 hour during a temperature increase of 45°. Water was then introduced with a temperature/time gradient increase of 15°/h until the isocyanate modification stated in Table 1 had been established.

### Comparative Example 1

In contrast to Examples 1-3, water was added with a temperature/time gradient increase of 3°/h until the isocyanate modification stated in Table 1 had been established.

TABLE 1

| | Comparison | Example | Example | Example |
|---|---|---|---|---|
| | 1 | 1 | 2 | 3 |
| TDI [kg] | 49.1 | 48.7 | 49.1 | 49.5 |
| Acidic substance | 50 | 50 | 50 | 50 |
| [g] | | | | |
| Water [kg] | 0.9 | 1.3 | 0.9 | 0.5 |
| NCO content | 40.5 | 31.4 | 37.2 | 42.2 |
| [% by weight] | | | | |
| Viscosity (25° C.) | 19 | 4.359 | 100 | 14 |
| [mPa · s] | | | | |
| Isocyanate | 16.0 | 34.8 | 22.8 | 12.4 |
| modification | | | | |
| [%] | | | | |
| Iodine color number | Not | 2.3 | 1.0 | 0.8 |
| | determined. | | | |
| | Product | | | |
| | was opaque; | | | |
| | incomplete | | | |
| | reaction | | | |

### Example 4

49.1 kg of TDI 80 (BASF: Lupranat® T 80 A) and 50 g of bis(2-ethylhexyl) phosphate were initially taken together at 65° C. 900 g of water were then introduced with a temperature/time gradient increase of 15°/h until the isocyanate modification stated in Table 2 had been established. Table 2 shows the trend in the shelf-life at 0° C., 25° C., and 40° C. over a period of 12 weeks.

TABLE 2

| Storage | Example 4 | | | | | | |
|---|---|---|---|---|---|---|---|
| tempera- | 1 day | 1 week | | | 12 weeks | | |
| ture | | ° C. | 25° C. | 40° C. | 0° C. | 25° C. | 40° C. |
| NCO con- | 37.4 | 37.3 | 37.3 | 37.2 | 37.2 | 37.1 | 37.1 |
| tent [%] | | | | | | | |
| Viscosity | 108 | 114 | 114 | 114 | 104 | 107 | 116 |
| 25° C. | | | | | | | |
| [mPa · s] | | | | | | | |
| Isocyanate | 22.4 | 22.6 | 22.6 | 22.8 | 22.8 | 23.0 | 23.0 |
| modifica- | | | | | | | |
| tion | | | | | | | |
| [%] | | | | | | | |

### Example 5

An isocyanate mixture was prepared from

50 parts by weight of the novel highly functional aromatic polyisocyanate according to Example 2,

30 parts by weight of TDI 80 (BASF: Lupranat® T 80 A) and

20 parts by weight of PMDI (BASF: Lupranat M 20 A)

by homogenization at room temperature in a nitrogen-applying blanketed stirred reactor. Table 3 shows the trend in the shelf-life at 0° C., 25° C. and 40° C. over a period of 12 weeks.

TABLE 3

| Storage | Example 5 | | | | | | |
|---|---|---|---|---|---|---|---|
| tempera- | 1 day | 1 week | | | 12 weeks | | |
| ture | | ° C. | 25° C. | 40° C. | 0° C. | 25° C. | 40° C. |
| Free NCO | 39.9 | 39.7 | 39.7 | 39.6 | 39.7 | 39.7 | 39.7 |
| [%] | | | | | | | |
| Viscosity | | | | | | | |
| 25° C. | 22 | 22 | 22 | 22 | 20 | 20 | 21 |
| [mPa · s] | | | | | | | |

### Examples 6 and 7, Comparative Example 2

Flexible polyurethane foams based on the starting components shown in Table 4 were produced. In Examples 6 and 7, modified polyisocyanate according to Example 2 is used.

TABLE 4

| | | |
|---|---|---|
| Comparative | | |
| Example 4 | Example 6 | Example 7 |

| | | |
|---|---|---|
| A component | Polyether alcohol 1 | 47.43 parts by weight |
| | Polyether alcohol 2 | 46.00 parts by weight |
| | Cell opener | 1.80 parts by weight |
| | Stabilizer | 0.60 part by weight |
| | Catalysts 1 | 0.51 part by weight |
| | Catalyst 2 | 0.11 part by weight |
| | Crosslinking agent | 0.30 part by weight |
| | Water | 3.10 parts by weight |

| | | | |
|---|---|---|---|
| B component | TDI 80: | TDI 80: | TDI 80: |
| | PMDI = | PMDI: | modified |
| | 62:38 | modified | polyisocyanate = |
| | parts by | polyisocyanate = | 39:61 |
| | weight | 52:20:28 | parts by weight |
| | | parts by weight | |
| Density [g/l] | 48 | 48 | 47 |
| Mechanical | | | |
| characteristics: | | | |
| Elongation at | 110 | 105 | 120 |
| break [%] | | | |
| Tensile strength | 175 | 175 | 200 |
| [kPa] | | | |
| Tear propagation | 0.57 | 0.64 | 0.76 |
| strength [N/mm] | | | |
| Comparative | 7 | 8 | 9.3 |
| strength [kPa] | | | |
| DS 50%, at 70° C. | 3 | 4 | 4.6 |
| and 22 h [%] | | | |
| Resilience [%] | 66 | 65 | 60 |
| Indentation | 410 | 490 | 540 |
| hardness [N] | | | |

CS compression set
Polyether alcohol 1-Trifunctional polyether alcohol based on glycerol/propylene oxide/ethylene oxide, OH number 35 mg KOH/g;
Polyether alcohol 2-Polymeric polyether alcohol based on glycerol/propylene oxide/ethylene oxide, acrylonitrile:styrene ratio = 1:1, solids content 30%, OH number 27 mg KOH/g;
Cell opener-Trifunctional polyether alcohol based on glycerol/propylene oxide/ethylene oxide, OH number 42 mg KOH/g;
Stabilizer-Tegostab B 4690;
Catalyst 1-Dabco 33 LV;
Catalyst 2-Niax A 1;
Crosslinking agent-Trifunctional alcohol based on glycerol, OH number 1740 mg KOH/g.

We claim:

1. A process for the preparation of highly functional aromatic polyisocyanates by reacting aromatic polyisocyanates, if required as a mixture with further mono- and/or polyisocyanates, with addition of catalytic acidic substances and water, wherein the polyisocyanates used comprise at least one tolylene diisocyanate and the catalytic acidic substance used is at least one alkyl and/or aralkyl phosphate, and the process is carried out in such a way that the water is added with a temperature/time gradient increase from 5 to 60°/hour and until an isocyanate modification of the starting NCO terminal groups of from 1 to 80% is established.

2. A process as claimed in claim 1, wherein the tolylene diisocyanate is used in industrially available isomer ratios, if required as a mixture with further aromatic mono- and/or polyisocyanates and/or aliphatic mono- and/or polyisocyanates.

3. A process as claimed in claim 1 or 2, wherein the catalytic acidic substance used is bis(2-ethylhexyl)phosphate.

4. A process as claimed in any of claims 1 to 3, wherein the water is used in the form of free water, steam, a substance releasing water of crystallization and/or a water-eliminating compound.

5. A process as claimed in any of claims 1 to 4, wherein the tolylene diisocyanate or the isocyanate mixture and the catalytic acidic substances are initially taken and water is then metered in with a temperature/time gradient increase from 9 to 40°/hour and to an isocyanate modification of the starting NCO terminal groups of from 8 to 60%.

6. A process as claimed in any of claims 1 to 5, wherein the tolylene diisocyanate or the isocyanate mixture or the catalytic acidic substances are initially taken, the respective other component is then metered in within from 0.01 to 2.5 hours with a temperature increase of up to 1000 and the water is then added.

7. A process as claimed in any of claims 1 to 6, wherein the total amount of catalytic acidic substances used is from 0.001 to 3.0% by weight, based on the weight of the total formulation.

8. A process as claimed in any of claims 1 to 7, wherein the total amount of water used is from 0.005 to 0.03 g/g of polyisocyanate.

9. A highly functional aromatic polyisocyanate which can be prepared as claimed in any of claims 1 to 8.

10. A highly functional aromatic polyisocyanate as claimed in claim 9, containing from 15 to 95 parts by weight of the modified highly functional polyisocyanate and from 85 to 5 parts by weight of tolylene diisocyanate and, if required, monomers and/or polymers of the isocyanate mixture used.

11. A highly functional aromatic polyisocyanate as claimed in claim 9 or 10, containing not more than 8 parts by weight of urea and higher molecular weight oligomers and not more than 2 parts by weight of uretdione and higher molecular weight oligomers.

12. A highly functional aromatic polyisocyanate as claimed in any of claims 9 to 11, which contains from 35 to 39% by weight of free NCO groups and has a viscosity of from 3 to 6000 mPa•s at 25° C., and a mean functionality of from 2.6 to 3.2, an average molecular weight of from 300 to 600 g/mol and an iodine color number of not more than 10.

13. The use of a highly functional aromatic polyisocyanate as claimed in any of claims 9 to 12 for the production of polyurethane foams, in particular flexible polyurethane foams.

14. A process for the production of flexible polyurethane foams by reacting organic and/or modified organic polyisocyanates (a) with, compounds (b) having hydrogen atoms reactive toward isocyanates, in the presence of water and/or other blowing agents (c), catalysts (d) and, if required, further assistants and additives (e), wherein the polyisocyanates (a) contain at least one highly functional aromatic polyisocyanate as claimed in any of claims 9 to 12.

15. A flexible polyurethane foam which can be prepared as claimed in claim 14, wherein said foam has a density of from 30 to 60 kg/m³ in combination with a total water content of from 2 to 8 parts by weight and an isocyanate index of from 75 to 115.

16. The use of a flexible polyurethane foam as claimed in claim 15 in the automotive industry and furniture industry.
